(19) United States
(12) Patent Application Publication
(10) Pub. No.: US 2014/0083948 A1
(43) Pub. Date: Mar. 27, 2014

(54) MAGNETIC PARTICLE SCAVENGING DEVICE AND METHOD

(71) Applicant: Guisheng Yang, Toronto (CA)

(72) Inventor: Guisheng Yang, Toronto (CA)

(21) Appl. No.: 14/004,663

(22) PCT Filed: Mar. 9, 2012
(86) PCT No.: PCT/CA2012/000198

§ 371 Date: Dec. 6, 2013

Related U.S. Application Data
(60) Provisional application No. 61/451,808, filed on Mar. 11, 2011.

(51) Int. Cl. A61M 1/36; B03C 1/02

(52) U.S. Cl. CPC A61M 1/36; B03C 1/02
USPC 210/695; 210/222

(57) ABSTRACT

The present invention is directed to a device for removing magnetic particles from a liquid, the device comprising at least one container for holding a liquid containing magnetic particles; and at least one magnetic column for placing into the at least one container, wherein when the liquid comes into contact with the at least one magnetic column, the magnetic particles are attracted towards, and bind to, the at least one magnetic column such that when the liquid is separated from the at least one magnetic column, the magnetic particles are removed from the liquid.

### FIELD OF THE INVENTION

This invention relates to liquids containing magnetic particles. More specifically, this invention is directed to a novel device and method for removing magnetic particles from a liquid.

### BACKGROUND OF THE INVENTION

Throughout this application, various references are cited to describe more fully the state of the art to which this invention pertains. The disclosures of these references are hereby incorporated by reference into the present disclosure in their entirety.

Devices and methods for removing magnetic particles are known from U.S. Pat. No. 2,029,078; U.S. Pat. No. 3,567,026; U.S. Pat. No. 3,676,337 U.S. Pat. No. 3,902,994; U.S. Pat. No. 4,141,687; U.S. Pat. No. 4,554,088; U.S. Pat. No. 4,663,029; U.S. Pat. No. 5,108,933; U.S. Pat. No. 5,200,084; U.S. Pat. No. 5,466,574; U.S. Pat. No. 5,622,831; U.S. Pat. No. 6,451,207; U.S. Pat. No. 6,468,810; U.S. Pat. No. 6,695,004; and U.S. 2006/0286137. Many of these devices and methods use external magnets that are not separated from the liquid into which they are inserted. Additionally, many of these devices and methods are only suitable for small volumes in experimental assays and are not suitable for larger scale volumes.

While the aforementioned devices are generally useful, it is desirable to provide a device and method that overcomes at least one deficiency of the prior art and further provides other advantages to the device itself.

### SUMMARY OF THE INVENTION

The present invention is a novel device and method for scavenging or removing magnetic particles from a liquid medium. The magnetic particles can be of any size, shape and configuration. For example, the magnetic particles can be, but are not limited to, filings, chippings, shavings etc. The devices and methods described herein find use in treating blood-borne diseases such as leukemia, diabetes, or viral infections. The devices and methods described herein are also useful in removing pollutants or contaminants from liquids other than blood or blood products such as, for example, bone marrow, cerebral spinal fluid (CSF), cell culture medium, a food, a milk, a beverage, reagents, oils, such as, for example, engine oil, lubricants such as, for example, machine lubricants, buffers, solvents, such as, for example, water, ethanol, formamide, phenol, chloroform, and other chemical liquids and chemical reagents. Other uses include removing DNA or RNA from solutions in experimental assays. The proteins can be enzymes, antibodies, receptors, polypeptides, haptens etc. The polypeptides can be polypeptide hormones. The haptens can be low molecular compounds, such as lectins, hormones, drugs, pesticides, toxins etc.

According to an aspect of the present invention there is provided a device for removing magnetic particles from a liquid, the device comprising: a container for holding the liquid and a magnetic column. The container can be any container suitable for holding the liquid and would be known by persons skilled in the art. In one embodiment of the present invention, the container is a tube. In another embodiment of the present invention, the container is at least one well in a plate. In another embodiment of the present invention, the at least one well is a plurality of wells. In an embodiment of the present invention, the pate is a plastic plate. The container and the magnetic column are sufficient for removing magnetic particles in a small volume of liquid (see (26) of FIG. 2). The magnetic column can be of any size, shape, and configuration. For example, the magnetic column can be, but is not limited to, a cake, a column, a needle, a bead, a nail, a scalpel, a spoon etc. Any size of magnetic column can be used individually to insert into a container to remove magnetic particles with or without mounting to a shaft. In one embodiment of the present invention, the container is non-magnetic.

According to an aspect of the present invention there is provided a device for removing magnetic particles from a liquid, the device comprising: a container for holding the liquid; a shaft; and at least one magnetic column mounted for movement about the shaft, wherein the magnetic column stirs the liquid and attracts the magnetic particles in the liquid. The movement about the shaft can be any movement as would be understood by persons skilled in the art. In one embodiment of the present invention, the movement about the shaft is multidirectional. In one embodiment of the present invention, the movement about the shaft is selected from the group consisting of stirring, rotation, vibration, swinging, circling, moving back and forth, up and down and combinations thereof.

In an aspect, the magnetic column is hollow and comprises an internal magnet. In an aspect, the magnet is removable from the magnetic column. In one embodiment of the present invention, the magnet is a permanent magnet. In another embodiment of the present invention, the magnet is an electromagnet. The magnetic column may further comprise a non-magnetic spacer and a removable cover. The outside of the magnetic column can be manufactured with some nail-like or network projections so that more substances can be held to it. In addition, the magnetic column can also be manufactured with hooks or other shapes (such as a knife or a spoon) in order to be more effective in holding substances.

In another aspect, a plurality of the magnetic columns is supported on the shaft in at least one array and each of the magnetic columns may be a different size and/or diameter. In an embodiment of the present invention, the at least one array is a single array supported on the shaft. In another embodiment of the present invention, the at least one array is a plurality of arrays supported on the shaft. In another embodiment of the present invention, the at least one array is any number of arrays as would be understood by persons skilled in the art. In another embodiment of the present invention, the at least one array is from 1 to about 20 arrays.

In another aspect, a plurality of arrays is supported on the shaft in an arrangement selected from the group consisting of substantially parallel substantially crossed (X or +) and combinations thereof.

In another aspect, the movement of about the shaft is selected from the group consisting of manual, automated and combinations thereof.

In another aspect, the liquid is selected from blood, a blood product, bone marrow, cerebral spinal fluid (CSF), cell culture medium, a food, a milk, a beverage, an oil, such as, for example, engine oil, lubricants such as, for example, that taken from a machine, buffers, solvents including, but not limited to, water, ethanol, formamide, phenol, chloroform, and other chemical liquids and chemical reagents. In an aspect, the liquid is blood or a blood product.

In another aspect, the magnetic particles are bound to cells, bacteria, algae, viruses, proteins, nucleic acids, or pollutants. When magnetic particles are used as solid supports, or the particles are larger than the targeted substances, the cells, bacteria, algae, viruses, proteins can be bound to the particles, instead of the particles being bound to the viruses, cells or proteins. However, once they are bound, there is no difference as they become complexes. When the particles are smaller than the targeted substances, the particles (nano-particles) are bound to the targeted substances. Whether the particles are bound to the targeted substances or the targeted substances are bound to the particles depends on the different situations. The magnet column then attracts the particle-cell/virus complexes to it.

The liquid could be a small or large volume as would be understood by persons skilled in the art. In an embodiment of the present invention, the volume is from about 10 μl to about 10⁶ liters. For example, for research use, the volume could be as small volume as about 10 μl. In an embodiment of the present invention, the volume is about 0.1 ml. For industrial use, the volume could be as large as the volume of a swimming pool. In one embodiment where the magnet is expanded to a large size, the scavenger can be manufactured as a vacuum cleaner and can be walked around the swimming pool so that the human hairs, algae, and other foreign (impurity) substances in the water can be removed. In one embodiment, the liquid is from about 300 ml to about 1000 ml. This volume may be used for clinical purposes.

According to another aspect of the present invention, there is provided a method of removing magnetic particles from a liquid, the method comprising: moving a magnetic column in the liquid to thereby attract the magnetic particles; and removing the magnetic column and attracted magnetic particles from the liquid. In an embodiment of the present invention, the moving is selected from the group consisting of stirring, rotation, vibration, swinging, circling, moving back and forth, up and down and combinations thereof. In an embodiment of the present invention, the moving is stirring.

In an aspect, the magnetic column is hollow and comprises an internal magnet and the magnet may be removable from the magnetic column. In another aspect, the magnetic column further comprises a non-magnetic spacer and a removable cover.

In another aspect, a plurality of the magnetic columns is supported on the shaft in at least one array and each of the magnetic columns is a different size/length and/or diameter. In an embodiment of the present invention, the at least one array is a single array supported on the shaft. In another embodiment of the present invention, the at least one array is a plurality of arrays supported on the shaft. In another embodiment of the present invention, the at least one array is any number of arrays as would be understood by persons skilled in the art. In another embodiment of the present invention, the at least one array is from 1 to about 20 arrays.

In another aspect, a plurality of arrays is supported on the shaft in an arrangement selected from the group consisting of substantially parallel (=), substantially crossed (X or +) and combinations thereof.

In another aspect, the movement of about the shaft is selected from the group consisting of manual, automated and combinations thereof.

In an aspect, the liquid is selected from the group consisting of blood, a blood product, bone marrow, CSF, cell culture medium, a food, a milk, a beverage, an oil, such as, for example, an engine oil, lubricants such as, for example, that taken from a machine, buffers, solvents including, but not limited to, water, ethanol, formamide, phenol, chloroform, other chemical liquids, other chemical reagents and combinations thereof. In an aspect, the liquid is selected from the group consisting of blood, a blood product and combinations thereof.

In another aspect, the magnetic particles are bound to cells, bacteria, algae, viruses, proteins, nucleic acids, pollutants or combinations thereof.

In another aspect, the liquid is a large volume as would be understood by persons skilled in the art. In an embodiment of the present invention, the liquid is in a volume from about 300 ml to about 1000 ml.

According to another aspect of the present invention, there is provided a device for removing magnetic particles from a liquid, the device comprising: a chamber comprising an inflow conduit and an outflow conduit; and a magnet supported within the chamber between the inflow conduit and the outflow conduit, wherein the magnet attracts the magnetic particles in the liquid when the liquid flows from the inflow conduit to the outflow conduit.

In an aspect, the magnet is stationary.

In an aspect, the magnet can be in any shape or size as would be understood by persons skilled in the art.

In an aspect, the magnet can be installed on the inside and/or outside of the wall of the chamber.

In another aspect, the device further comprises a plurality of holding portions for supporting the magnet within the chamber.

In another aspect, the magnet comprises a protective coating.

In another aspect, the device comprises two portions that engage one another to form the chamber, one portion comprising the inflow conduit and another portion comprising the outflow conduit and the magnet.

In an aspect, the magnet is removable from the chamber and the two portions engage one another by screwing together.

In another aspect, the outer diameter of the magnet is smaller than the inner diameter of the chamber. In another aspect, the magnet comprises an aperture through which the liquid flows. In another aspect, the magnet is concave on one or both sides.

In an aspect, the liquid is selected from the group consisting of blood, a blood product, bone marrow, CSF, cell culture medium, a food, a milk, a beverage, an oil, such as, for example, an engine oil, lubricants such as, for example, that taken from a machine, buffers, solvents including, but not limited to, water, ethanol, formamide, phenol, chloroform, other chemical liquids, other chemical reagents, and combinations thereof. In an aspect, the liquid is selected from the group consisting of blood, a blood product and combinations thereof.

In an aspect, the magnetic particles are bound to cells, bacteria, algae, viruses, proteins, nucleic acids, or pollutants.

In another aspect, the liquid is a large volume as would be understood by persons skilled in the art. In an embodiment of the present invention, the liquid is in a volume from about 300 ml to about 1000 ml.

According to another aspect of the present invention, there is provided a method of removing magnetic particles from a liquid, the method comprising: passing the liquid into a drip chamber comprising an internal magnet such that the liquid contacts and flows past the magnet, the magnet attracting the magnetic particles in the liquid; and passing the liquid out of the drip chamber.

In an aspect, the drip chamber comprises a plurality of holding portions for supporting the magnet within the chamber.

In another aspect, the magnet comprises a protective coating.

In another aspect, the drip chamber comprises two portions that engage one another to form the drip chamber, one portion comprising an inflow conduit and another portion comprising an outflow conduit and the magnet. In an aspect, the magnet is removable from the drip chamber and the two portions engage one another by screwing together.

In an aspect, the outer diameter of the magnet is smaller than the inner diameter of the drip chamber. In another aspect, the magnet comprises an aperture through which the liquid flows. In another aspect, the magnet is concave on one or both sides.

In another aspect, the liquid is selected from blood, a blood product, bone marrow, CSF, cell culture medium, a food, a milk, a beverage, an oil, such as, for example, an engine oil, lubricants such as, for example, that taken from a machine, buffers, solvents including, but not limited to, water, ethanol, formamide, phenol, chloroform, and other chemical liquids and chemical reagents. In an aspect, the liquid is blood or a blood product.

In another aspect, the magnetic particles are bound to cells, bacteria, algae, viruses, proteins, nucleic acids, or pollutants.

In another aspect, the liquid is a large volume, such as from about 300 ml to about 1000 ml.

According to another aspect of the present invention, there is provided a method of treating a blood-borne disease or disorder in a subject, the method comprising: treating blood of the subject with magnetic particles targeted to bind to the disease- or disorder-causing moiety; and removing the magnetic particles and disease- or disorder-causing moiety from the blood by using the device described herein.

In an aspect, the blood-borne disease or disorder is selected from a cancer, a virus, and an autoimmune disease. In an aspect, the cancer is leukemia; the virus is HIV, HBV, or HCV; rotavirus and the autoimmune disease is diabetes, systemic lupus erythematosus, or rheumatoid arthritis.

In another aspect, the disease- or disorder-causing moiety is selected from a cell, a viral particle, an autoimmune protein complex, a toxic agent, a protein complex, and a cholesterol complex.

In another aspect, the blood is removed from the subject for treatment and returned to the subject after treatment.

According to another aspect of the present invention, there is provided a use of the device described herein for treating a blood-borne disease or disorder in a subject, wherein magnetic particles targeted to bind to the disease- or disorder-causing moiety are present in the blood of the subject.

In an aspect, the blood-borne disease or disorder is selected from a cancer, a virus, and an autoimmune disease. In an aspect, the cancer is leukemia; the virus is HIV, HBV, or HCV; and the autoimmune disease is diabetes, systemic lupus erythematosus, or rheumatoid arthritis.

In another aspect, the disease- or disorder-causing moiety is selected from a cell, a viral particle, an autoimmune protein complex, a toxic agent, a protein complex, and a cholesterol complex.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from the detailed description.

### BRIEF DESCRIPTION OF THE DRAWINGS

Embodiments will now be described, by way of example only, with reference to the attached figures, wherein:

FIG. 1 is perspective view of a device of the present invention;

FIG. 2 is a perspective view of the columns of the device of FIG. 1;

FIG. 3 is top plan view and side elevation view of the arrays of the device of FIG. 1;

FIG. 4 is a perspective view of the device of FIG. 1 showing its assembly;

FIG. 5a is a side elevation view of another device of the present invention;

FIG. 5b is a side cross-section view of the device of FIG. 5a;

FIG. 5c is a side cross-section view of the device of FIG. 5a in use;

FIG. 6a is a side elevation view of the device of FIG. 5a when disassembled;

FIG. 6b is a cross-section view of the device of FIG. 6a;

FIG. 6c is a top cross-section view of the device of FIG. 5a;

FIG. 7a is an illustration of a method of use of a device of the present invention;

FIG. 7b is an illustration of a method of use of a device of the present invention;

FIG. 8 is an illustration of the binding of targeted particles to magnetic particles to form a complex and the binding of the complex to a magnet in accordance with an aspect of the present invention;

FIG. 9 is a perspective view of a device of the present invention.

### DETAILED DESCRIPTION OF THE INVENTION

The present invention is directed to novel devices and methods for removing or scavenging magnetic particles from a liquid medium. These devices and methods find use in removing magnetic particles from biological liquids such as from blood, a blood product, bone marrow, CSF, cell culture medium, a food, a milk, a beverage, an oil, such as, for example, an engine oil, lubricants such as, for example, that taken from a machine, buffers, solvents including, but not limited to, water, ethanol, formamide, phenol, chloroform, and other chemical liquids and chemical reagents. The magnetic particles may themselves be contaminants or pollutants in the liquid or they may be bound to a contaminant or disease-causing moiety in the liquid. Alternatively, the magnetic particles may be a desirable component of the liquid that must be removed from the liquid for purification.

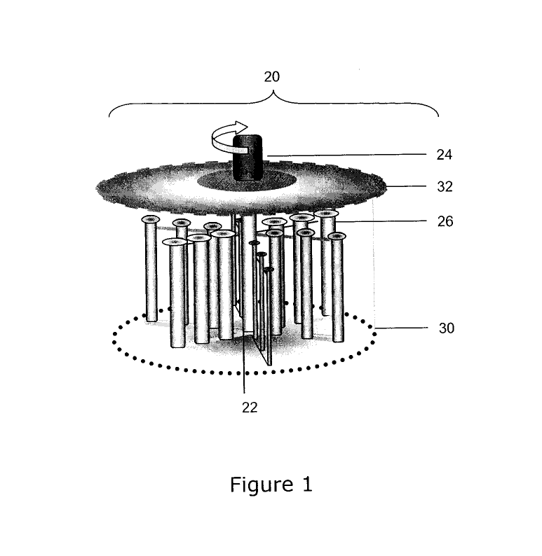

The invention is now herein described with reference to FIG. 1, which shows one aspect of the device of the present invention. This device 20 comprises a shaft 22 that is connected to a knob 24. The shaft 22 and the knob 24 are connected to one another such that movement of the knob 24, in this embodiment rotation, causes a corresponding movement, in this embodiment, rotation of the shaft 22. In this way, the device 20 can be operated manually and/or automatically through movement, in this embodiment rotation, of the knob 24.

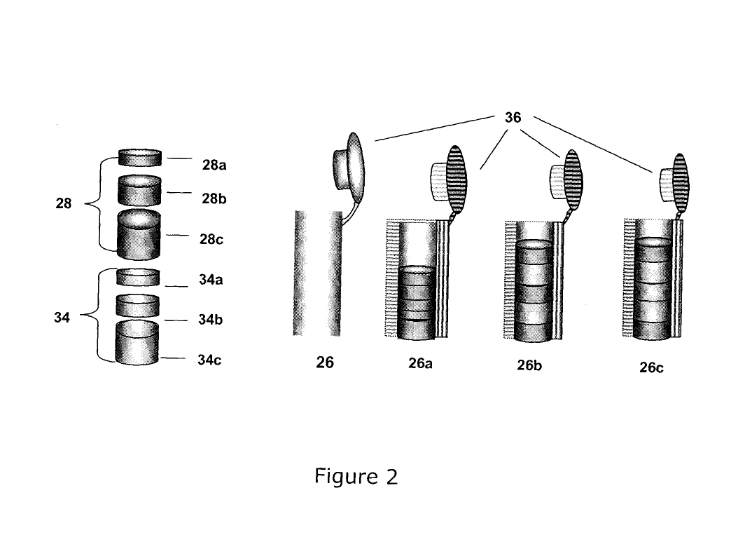

Mounted to the shaft 22 is a plurality of columns 26. The columns 26 are hollow, house magnets 28 (see FIG. 2), and are rotational about the shaft 22. The columns 26 are supported about the shaft in arrays of six columns each in the illustrated embodiment. Six columns and three arrays is merely an example. The columns and the arrays may be more or fewer according to the diameter of the device 20 as would be understood by persons skilled in the art. The length of the columns 26 may be shorter or longer according to the deepness or shallowness of the liquid volume inside the container 30 as would be understood by persons skilled in the art. Each of the arrays comprises different sized columns 26. The variation in sizes allows a corresponding variation in strength of the magnetic field, which permits customization of the device 20 by the end user as desired. The shaft 22 and columns 26 are insertable into a container 30 for holding liquid and the container may be closed using a cover 32.

It will be understood that the device 20 is multi-functional in that it may act as a stirrer when magnets 28 are not housed in the columns 26, whereas it can additionally act to attract magnetic particles in a liquid when magnets 28 are housed in the columns 26.

Turning now to FIG. 2, the columns 26 and magnets 28 are shown in isolation. The magnets 28 are shown in three different sizes 28a, 28b, and 28c. Additionally, non-magnetic spacers 34 are shown in three different sizes 34a, 34b, and 34c. The non-magnetic spacers may be made of any non-magnetic material, such as metal (aluminum, lead or copper, for example), porcelain, glass, ceramics, plastic, or wood. By combining these magnets 28 and spacers 34 in different permutations, the resulting magnetic field may be adjusted to meet the needs of the end user. For example, column 26a includes only magnets 28 and produces a strong magnetic field. Column 26b includes three magnets 28 separated by one spacer 34 each, creating an intermediate magnetic field at three different planes within the container 30. Finally, column 26c includes two magnets 28 separated by three spacers 34, creating a weaker magnetic field at two different and distant planes within the container 30. This combinatorial arrangement of magnets 28 and spacers 34 allows for a near limitless customization of the magnetic field by the end user.

This combinatorial arrangement is advantageous because any magnetic particles in the liquid may float for some time rather than settle to the bottom of the container 30 immediately due to their different specific gravities. By adjusting the plane of the magnetic field, the magnetic particles may be attracted to the magnetic columns 26 immediately rather than waiting for the particles to sink in the liquid. Additionally, the size of the magnets may be adjusted in order to create a stronger or weaker magnetic field, depending upon the concentration or size of the magnetic particles in the liquid.

From FIG. 2 it will be evident that the columns 26 are tubes having one closed end and one open end into which the magnets 28 and spacers 34 may be inserted. The open end may be protected by a lid 36, thereby preventing liquid in the container 30 from contaminating the magnets 28 and spacers 34. In this way, the magnets 28 and spacers 34 are reusable without necessarily requiring cleaning between each use.

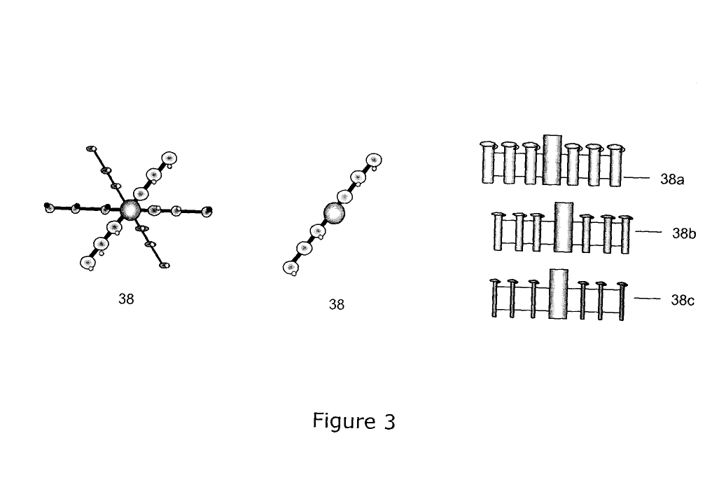

FIG. 3 illustrates the arrangement of the columns 26 into arrays 38. As is shown in FIGS. 1 and 2, three arrays 38 containing six columns each, for example, may be assembled about the shaft 22. Alternatively, a single array 38 or any number of arrays may be assembled about the shaft 22. The arrays 38 may be assembled about the shaft 22 in any arrangement as would be understood by persons skilled in the art. In an embodiment of the present invention, the arrays 38 are assembled about the shaft 22 in an arrangement selected from the group consisting of substantially parallel (=), substantially crossed (X or +) and combinations thereof. FIG. 3 illustrates the assembling of the arrays 38 about the shaft 22 in a substantially crossed arrangement. The arrays 38 may contain columns 26 of different sizes or diameters, creating large 38a, medium 38b, and small 38c arrays. These arrays 38a, 38b, and 38c may be assembled about the shaft in any combination and each of the arrays 38 need not contain only one type of column 26 as is shown in the Figures. It is contemplated that different columns 26 may be combined together in a single array 38. Thus, the end used is provided with even more customizability with respect to the magnetic field that is created.

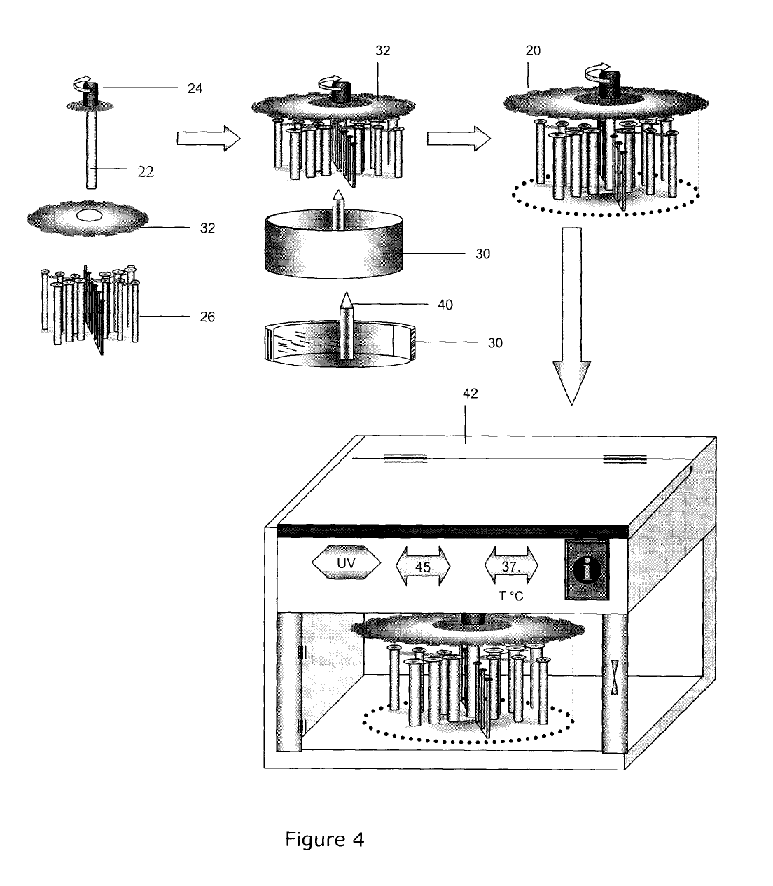

Turning now to FIG. 4, assembly of the device 20 is shown. The knob 24 and shaft 22 are inserted through a hole in the cover 32 and the shaft is attached to the arrays 38 containing magnets 28 and optionally spacers 34 in the desired configuration. The shaft 22 is hollow and can thus be placed on top of a post 40 that extends upwardly from the container 30. Alternatively, the shaft 22 can be placed or fixed on any spot where the various motions or movements can be accomplished as would be understood by persons skilled in the art. The shaft 22 is rotatable on the post 40 by using the knob 24. At this point, the device 20 may be operated manually by simply turning the knob 24, thereby causing the arrays 38 to spin within the container 30. Alternatively, the device 20 may be inserted into an automated housing 42 that may control the speed of rotation and other desired parameters, such as time, UV sterilization, temperature, and light source.

In FIG. 4, housing 42 is an example device for the medical use purpose. In use, the device 20 is assembled as described in a desired configuration using the magnets 28, spacers 34, and arrays 38. A liquid containing magnetic particles is placed into the container 30 and the knob 24 is turned. This causes stirring of the liquid and movement of the magnetic field throughout the liquid, thereby increasing the likelihood that the magnetic particles in the liquid will be found within the magnetic field and thus be attracted to the magnets 28. After a period of time, the liquid may be removed from the container 30 and/or the arrays 38 may be removed from the liquid, depending upon whether the purified liquid is the desired end product or whether the magnetic particles are the desired end product. The magnetic particles will be attracted to the columns and this attraction will not cease until the columns are demagnetized, for example, by removal of the magnets therein.

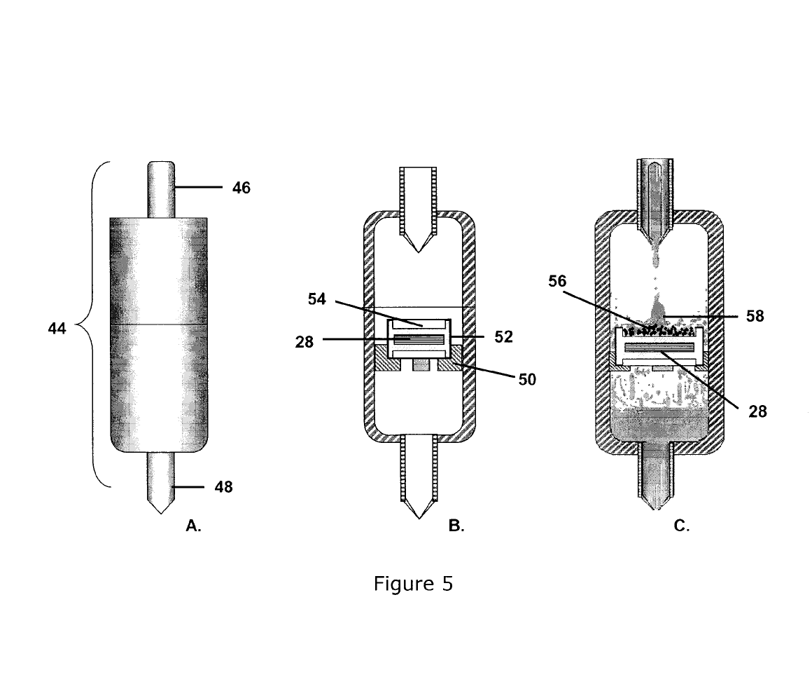

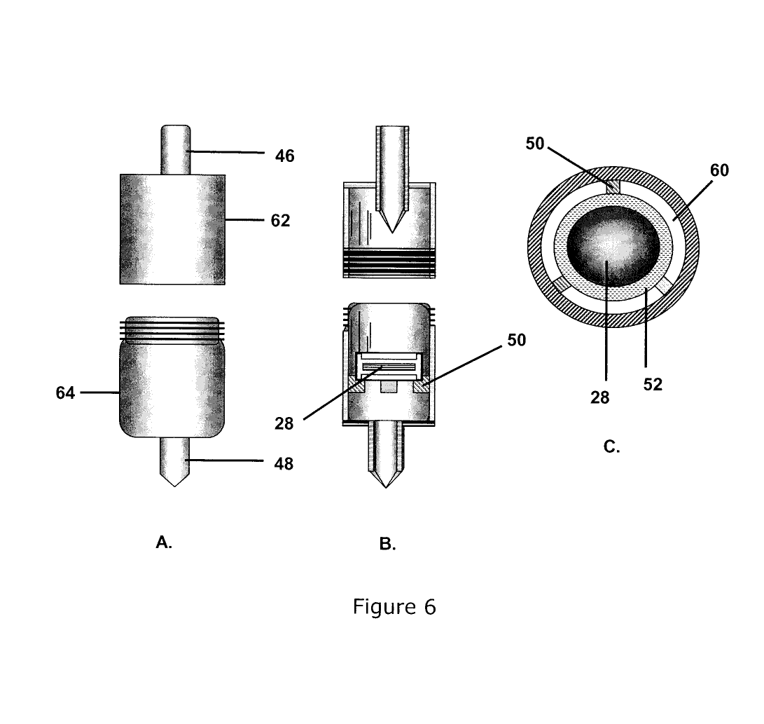

Turning now to FIGS. 5 and 6, another aspect of the device of the present invention is illustrated. Here, the device takes the form of a hollow drip chamber 44 having an inflow conduit 46 and an outflow conduit 48 through which liquid 58 may flow. A magnet 28 is supported within the drip chamber 44 on holding portions 50. The outside diameter of the magnet 28 is smaller than the inside diameter of the drip chamber 44 so that the liquid 58 may flow past the magnet 28 through spaces 60 between the holding portions 50 and out through the outflow conduit 48. Alternatively, the magnet 28 may have the same diameter as the inside diameter of the drip chamber 44, however, in this case, there should be at least one aperture in the magnet 28 to allow the liquid 58 to flow therethrough. It will be understood that the magnet 28 in this aspect of the device is stationary and does not move with the liquid 58. Alternatively, the magnet 28 may be mounted to the wall, inside and/or outside of the chamber 44. The chamber 44 also can be modified to let the inflow conduit 46 and the outflow conduit 48 be connected to a vein or artery of a patient by implanting the device in the patient's body to capture disease causing moieties.

The drip chamber 44 is made from two portions that engage one another to form the drip chamber 44. One portion 62 comprises the inflow conduit 46, while the other portion 64 comprises the outflow conduit 48 and the magnet 28. As is shown in FIG. 6, the two portions 62, 64 screw together to form the drip chamber 44. It will be understood that the two portions could engage one another by methods other than screwing, such as by a friction fit or by snapping together, for example. Alternatively, the drip chamber 44 could be provided as a unitary device that does not come apart, having the magnet 28 manufactured therein. The drip chamber 44 may be transparent so that the dripping speed can be monitored. The material of drip chamber may be the same as the material of the syringe or the coating 52 materials.

In the illustrated embodiment, the magnet 28 has a protective coating 52. The material of the protective coating 52 may be consistent with that of the syringe or other medical use consumables. It should be a non-toxic, and regulatory approved grade material, such as, for example polyethylene or polythene. The thickness of the protective coating may be from about 0.2 to about 1.0 mm. The coating 52 does not interfere with the magnetic field produced by the magnet 28 and is present to prevent any adverse reaction from occurring between the magnet 28 and the liquid 58 with which it is in contact. For example, if the liquid 58 is blood, the coating 52 may be biocompatible and inert. Additionally, the coating 52 may be shaped so as to form concave wells 54 on either or both sides of the magnet 28. The wells 54 are formed on both sides of the magnet 28 so that the orientation in which that magnet 28 is placed in the drip chamber 44 does not matter. The wells 54 function to increase the contact time of the liquid with the magnetic field in order to help ensure that any magnetic particles 56 in the liquid 58 are attracted and held in place by the magnet 28 while the liquid 58 continues to flow past.

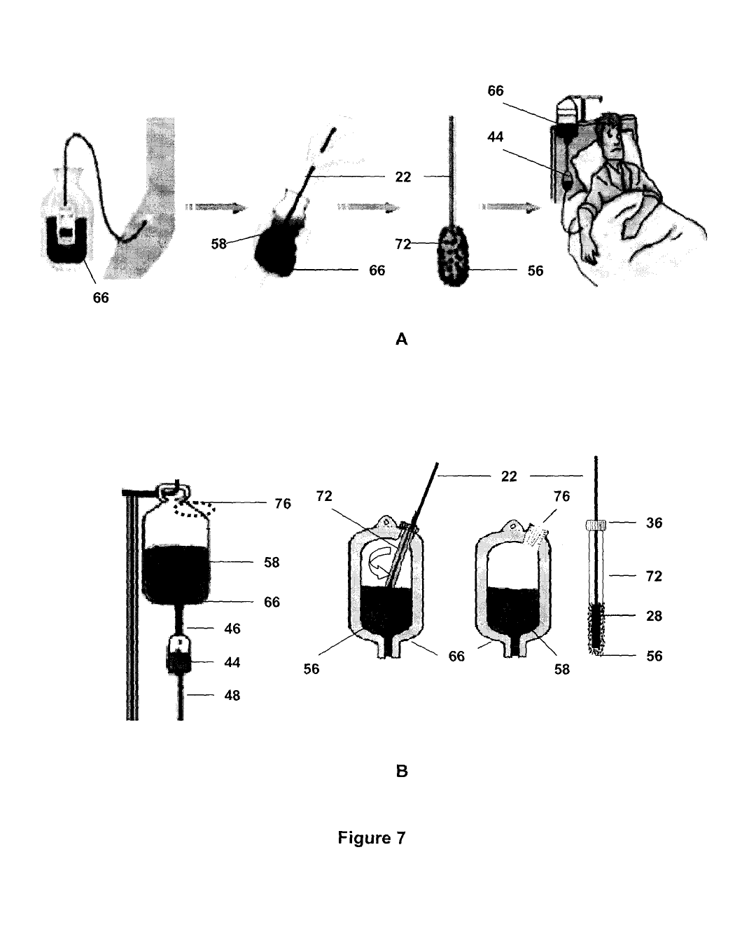

If the liquid 58 is blood or a blood product, the drip chamber 44 may be suspended below a medical liquid container 66 such as that shown in FIG. 7. In this embodiment, the blood or blood product flows from the medical liquid container 66 into the drip chamber 44 and eventually into the subject. In this way, the blood or blood product can be cleaned of magnetic particles 56 prior to its entry into the subject. The blood or blood product may be the subject's own blood or blood product that had been earlier removed. In one aspect, targeted magnetic particles 56 were bound to malignant cells, for example, found within the subject's blood. By passing this blood through the drip chamber 44, the malignant cells will be removed from the blood along with the magnetic particles 56 prior to returning the blood to the subject's system.

In use, a liquid 58, containing magnetic particles 56, is allowed to flow into the drip chamber 44 via the inflow conduit 46. The liquid 58 contacts the magnet 28 and any magnetic particles 56 found in the liquid are attracted to and held in place by the magnet 28 while the liquid 58 continues to flow past the magnet 28 and out through the outflow conduit 48. Upon exiting the drip chamber 44, the liquid 58 will be substantially free of magnetic particles 56.

The invention is now herein described with reference to FIGS. 7a and 7b, which show another aspect of the device of the present invention. In particular, FIG. 7a shows the device comprising a medical liquid container 66 and a magnetic column 72. In this particular embodiment, blood 58 is taken from a patient suffering from leukemia and collected in the medical liquid container 66. The leukemia cells in the collected blood 58 are bound by magnetic particles 56. The magnetic column 72 is submersed in the collected blood 58 in the medical liquid container 66 and swirled or stirred. The leukemia cells that are bound to the magnetic particles 56 in the blood 58 magnetically bind to the magnetic column 72 and are removed from the blood with the magnetic column when the magnetic column is removed from the medical liquid container 66. The leukemia cell-free blood can then be transfused back into the patient. Referring now to FIG. 7b, the medical liquid container 66 is a blood bottle with a drip-chamber 44. There is an opening 76 in the shoulder of the blood bottle through which the magnetic column 72 can be inserted. Once again, blood 58 is taken from a patient suffering from leukemia and collected in the blood bottle. The leukemia cells in the collected blood 58 are bound by magnetic particles 56. The magnetic column 72 is inserted through the opening 76 in the shoulder of the blood bottle and submersed in the collected blood 58 and swirled or stirred. The leukemia cells that are bound to the magnetic particles 56 in the blood 58 magnetically bind to the magnetic column 72 and are removed from the blood with the magnetic column when the magnetic column is removed from the blood bottle. The captured leukemia cells can then be placed into a separate container for analysis.

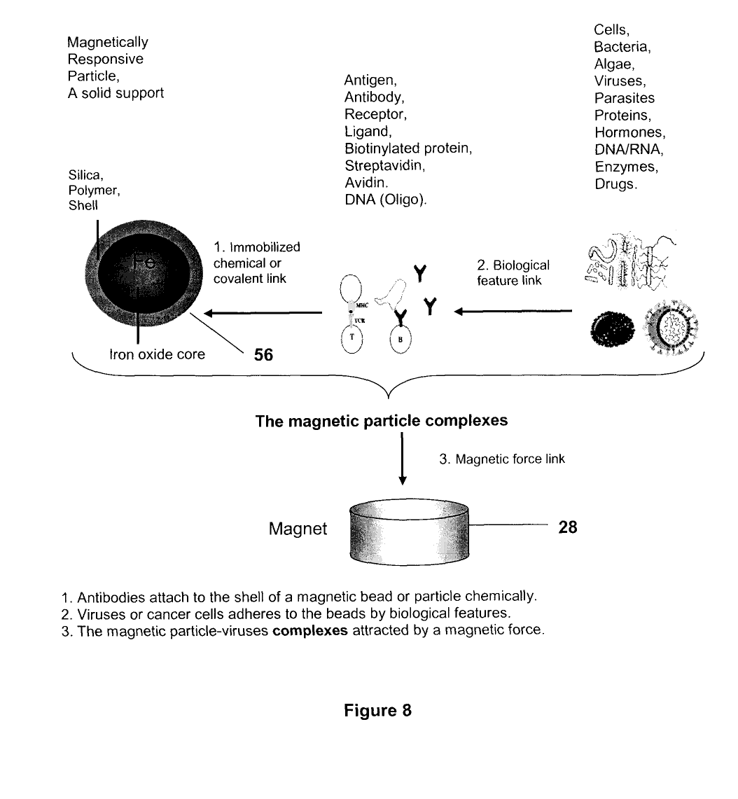

In an embodiment of the present invention, targeted substances can be made to bind to magnetic support material. The targeted substances can be members of any specific binding pair, such as, for example, a pair of bio-specific ligands and receptors, antigen and antibodies, or anything having specific binding affinities. The determination of any member of a bio-specific binding pair is dependent upon its selective interaction with the other member of the pair. For example, in forming an immune-complex, a “sandwich” is formed in which the “layers” are magnetic-particle/antigen/antibody or magnetic-particle/antibody/antigen. The sandwich can also be magnetic-particle/receptor/viruses or magnetic-particle/receptor/cells. Referring now to FIG. 8, magnetically responsive particles provide a solid support. In this particular embodiment, the magnetic particles are composed of an iron core, such as an iron oxide core, and a silica/polymer shell. The size range of the magnetic particles may be from about 10 nm to about 500 μm. The bio-affinity components are attached to the particle by covalent binding or by biotin/streptavidin coupling. The bio-affinity components are needed for the cells, viruses and other targeted substances to be attached to the particles, such as antigen-antibody, ligand-receptor, etc. In this particular embodiment, the particle is coated with silica or polymer so that it can provide a high surface area to present for example, more than one receptor and be surrounded by a few targeted substances, e.g. it can form flower-like complexes. For the same reason, if one cell has more than one receptor in the cell's membrane, more than one particle can be adhered to one cell according to the design. In this particular embodiment, the magnetic responsive particle itself is not magnetized. It plays the role of a carrier and behaves as a true colloid. It becomes magnetic only when it is subjected to a magnetic field. In addition, the coating materials can protect the, for example iron, of the particle from direct contact with the liquid in order to avoid certain chemical reactions between the, for example iron, of the particle with the liquid components. This can be a very important safety issue when the particles are used for clinical purposes.

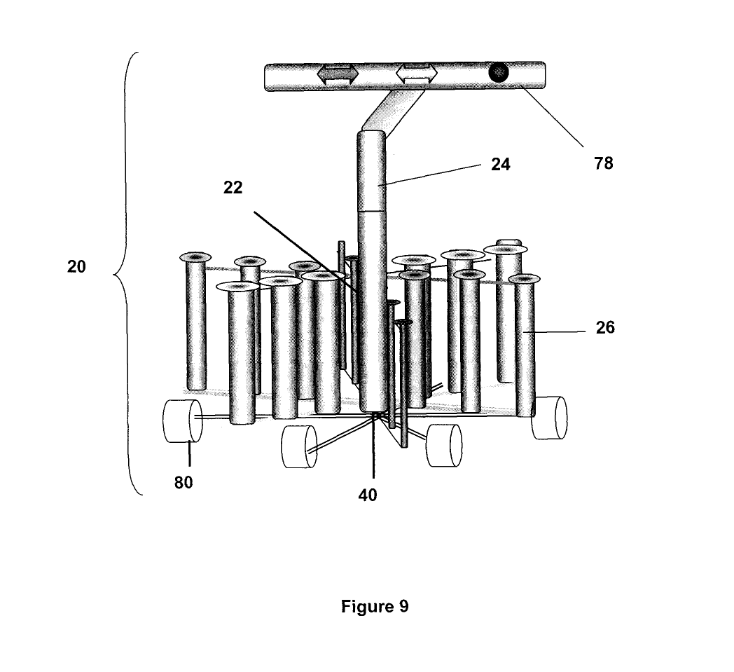

The invention is now herein described with reference to FIG. 9, which shows another aspect of the device of the present invention. In this particular embodiment, the device 20 is a scavenger used in industry, such as, for example, in the swimming pool industry. In this embodiment, where the magnet is expanded to a large size, the device 20 is a scavenger and is manufactured in the form of something like a vacuum cleaner to remove human hairs and other impurity substances from water in a swimming pool wherein the liquid container is the pool and the shaft 22, the knob 24, the columns 26, and the post 40, make up the device 20. The device 20 could be motorized or moved manually by pushing the handle 78 such that the device 20 moves through the water in a swimming pool, in this embodiment, over the interior surface of the swimming pool on wheels 80 supported by post 40, so that the human hairs, algae, and other foreign (impurity) substances in the water can be removed. This embodiment is more effective and economical than filter cleaning.

The devices described herein find use in methods of removing magnetic particles from a liquid. The type of liquid is non-limiting and some examples include blood, blood products, bone marrow, CSF, cell culture medium, foods, milk, beverages, an oil, such as, for example, an engine oil, lubricants, buffers, solvents including, but not limited to, water, ethanol, formamide, phenol, chloroform, and other chemical liquids and chemical reagents. The magnetic particles themselves may be desirable to remove from the liquid, or the magnetic particles may be bound to a component in the liquid that is desirable to remove. For example, the magnetic particles may be targeted to bind to cells, bacteria, algae, viruses, proteins, nucleic acids, or pollutants found in the liquid. In this way, the devices described herein may be used to clean polluted or contaminated water or to remove bits of metal scrapings found in engine oil and lubricants. Alternatively, the devices may be used to treat diseases or disorders such as cancer, including leukemia, viruses, including HIV, HBV, or HCV, or autoimmune diseases, including diabetes, systemic lupus erythematosus, or rheumatoid arthritis. These listed diseases and disorders are considered non-limiting, as any liquid disease or disorder (any disease or disorder that involves circulating cells, viruses, proteins, auto-antibodies in a bodily fluid, such as blood, bone marrow, CSF) may be treated using the presently claimed devices. The devices described herein also find use in experimental assays, such as in isolating proteins, bacteria, viruses, DNA or RNA from liquid solutions.

In treating such diseases or disorders, the magnetic particles are targeted to bind to the disease-causing moiety. For example, in the case of leukemia, the magnetic particles would be targeted to the malignant cells. In the case of a viral infection, the magnetic particles would be targeted to a viral particle. In the case of an autoimmune disorder, the magnetic particles would be targeted to an autoimmune protein complex. Other protein complexes or cholesterol complexes may be targeted in order to treat other diseases or disorders.

The description as set forth is not intended to be exhaustive or to limit the scope of the invention. Many modifications and variations are possible in light of the above teaching without departing from the spirit and scope of the following claims. It is contemplated that the use of the present invention can involve components having different characteristics. It is intended that the scope of the present invention be defined by the claims appended hereto, giving full cognizance to equivalents in all respects.

1.-91. (canceled)

92. A device for removing magnetic particles from a liquid, the device comprising:
a. at least one container for holding a liquid containing magnetic particles; and
b. at least one magnet for placing into the at least one container,
wherein when the liquid comes in contact with the at least one magnet, the magnetic particles are attracted towards, and bind to, the at least one magnet such that when the liquid is separated from the at least one magnet, the magnetic particles are removed from the liquid.

93. The device of claim 92, wherein the at least one magnet is a magnetic column.

94. The device of claim 93, further comprising at least one shaft upon which the at least one magnetic column is supported for movement thereabout.

95. The device of claim 93, wherein the at least one magnetic column is hollow and comprises an internal magnet.

96. The device of claim 95, wherein the internal magnet is selected from the group consisting of a permanent magnet, an electromagnet and combinations thereof.

97. The device of claim 95, wherein the internal magnet is removable from the at least one magnetic column.

98. The device of claim 93, wherein the at least one magnetic column further comprises a non-magnetic spacer.

99. The device of claim 93, wherein the at least one magnetic column comprises a removable cover.

100. The device of claim 94, wherein the at least one magnetic column is a plurality of magnetic columns.

101. The device of claim 100, wherein at least two of the magnetic columns of the plurality of magnetic columns have different dimensions.

102. The device of claim 100, wherein the plurality of magnetic columns is supported on the at least one shaft in at least one array.

103. The device of claim 102, wherein the at least one array is a plurality of arrays supported on the at least one shaft.

104. The device of claim 93, wherein the liquid is selected from the group consisting of blood, a blood product, bone marrow, CSF, cell culture medium, a food, a milk, a beverage, oils, lubricants, buffers, solvents selected from the group consisting of water, ethanol, formamide, phenol, chloroform, reagents, and combinations thereof.

105. The device of claim 93, wherein the magnetic particles are bound to cells, bacteria, algae, viruses, proteins, nucleic acids or pollutants.

106. The device of claim 93, wherein the container is a chamber comprising an inflow conduit and an outflow conduit; and the magnet is supported within the chamber between the inflow conduit and the outflow conduit, wherein the magnet attracts and binds the magnetic particles in the liquid when the liquid flows from the inflow conduit to the outflow conduit.

107. The device of claim 107, further comprising a plurality of holding portions for supporting the magnet within the chamber.

108. The device of claim 107, wherein the magnet comprises a protective coating.

109. The device of claim 107, comprising two portions that engage one another to form the chamber, one portion comprising the inflow conduit and another portion comprising the outflow conduit and the magnet.

110. The device of claim 107, wherein the magnet is removable from the chamber.

111. The device of claim 107, wherein the magnet is mounted to the wall of the chamber.

112. The device of claim 107, wherein the magnet comprises an aperture through which the liquid flows.

113. The device of claim 107, wherein the inflow conduit and the outflow conduit are configured to be connected to a vein or artery of a patient directly or indirectly.

114. A method of treating a blood-borne disease or disorder in a subject, the method comprising:
a. treating blood of the subject with magnetic particles targeted to bind to a moiety causing the disease or disorder; and
b. removing the magnetic particles and disease- or disorder-causing moiety from the blood by using the device of claim 92.

115. A method of removing magnetic particles from a liquid, the method comprising:
a contacting a liquid containing magnetic particles with at least one magnet in the liquid to thereby attract and bind the magnetic particles to the at least one magnet; and
b. separating the liquid from the at least one magnet and bound magnetic particles to remove the magnetic particles from the liquid.

116. The method of claim 115, wherein step a. comprises passing the liquid into a drip chamber comprising an internal magnet such that the liquid contacts and flows past the magnet, the magnet attracting and binding the magnetic particles in the liquid and step b. comprises passing the liquid out of the drip chamber.
